(19) United States
(12) Patent Application Publication
(10) Pub. No.: US 2005/0037992 A1
(43) Pub. Date: Feb. 17, 2005

(54) Composition and method for treating neurological disorders

(76) Inventors: John Lyons, London (GB); Lucy Chang, San Mateo, CA (US)

Correspondence Address: WILSON SONSINI GOODRICH & ROSATI, 650 PAGE MILL ROAD, PALO ALTO, CA, 943041050, US

(21) Appl. No.: 10/877,046

(22) Filed: Jun. 24, 2004

Related U.S. Application Data
(60) Provisional application No. 60/489,394, filed on Jul. 22, 2003.

(51) Int. Cl. A61K 31/7072

(52) U.S. Cl. 514/49; 514/50

(57) ABSTRACT

Compositions, kits and methods are provided for treating or preventing neurological disorders associated with aberrant silencing of gene expression by reestablishing the gene expression through inhibition of DNA methylation and/or histone deacetylase. The compositions and methods include administering to a patient suffering from the neurological disorder a therapeutically effective amount of a DNA methylation inhibitor, such as decitabine, preferably in combination with an effective amount of a histone deacetylase inhibitor. The compositions, kits and methods can be used to treat or present neurological disorders such as Lou Gehrig's disease, fragile X syndrome, Parkinson's disease and Alzheimer's disease.

### CROSS-REFERENCE

This application claims the benefit of U.S. Provisional Application No. 60/489,394, filed Jul. 22, 2003 which is incorporated herein by reference in its entirety.

### BACKGROUND OF INVENTION

This invention related to compositions and methods for treating and/or preventing neurological disorders using a DNA methylation inhibitor separately or in combination with a histone deacetylase inhibitor. Prophylactic treatment is a preferred method of treatment of neurological disorders given the limited ability of the central nervous system to regenerate neurons.

In the past, the clinical management of numerous neurological disorders has been frustrated by the progressive nature of degenerative, traumatic, or destructive neurological disorders and by the limited efficacy and serious side effects of available pharmacological agents. The complexity of preventing and treating neurological disorders is attributable in part to the fact that more genes are expressed in the nervous system that in any other tissue. Furthermore, the cytoarchitecture and cellular signaling mechanisms of the nervous system are very complex in nature.

As certain genes or genes variants that are desirable for the maintenance of normal healthy neurons are suppressed or inhibited, it is desirable to reestablishing and/or upregulating genes that associated with prevention of the neurological disorders or restoration of normal functions.

### SUMMARY OF THE INVENTION

The present invention provides new and improved compositions, kits, and methods for treating and preventing neurological disorders (e.g., ALS, Parkinson's disease, Alzheimer's disease, fragile X syndrome, etc.) by using a DNA methylation inhibitor separately or in combination with a histone deactylase (HDAC) inhibitor. It is believed that methylation of cytosine residues in DNA and removal of acetyl groups from histones are the two primary mechanisms for gene silencing. Due to methylation and/or histone deacetylation of neurotransmission-related genes, expression of certain genes required for normal neuronal functions and neurotransmission is suppressed or completely silenced. Inaction of these genes in the affected cells leads to neurodegeneration, which eventually results in one or more neurological disorders disclosed herein. The present invention provides an innovative approach for efficacious treatment of patients with such neurological disorders, preferably through a combination therapy of a DNA methylation inhibitor and an HDAC inhibitor. By using the combination therapy, transcription of the neurologically important genes can be reestablished, thereby regaining the functions that are lost due to transcriptional silencing of such genes by aberrant DNA methylation and/or deacetylation. Through such a combination treatment, a lower dosage of the inhibitors may be required for achieving a superior clinical outcome than by using a monotherapy involving either the DNA methylation inhibitor or the HDAC inhibitor alone. It may also be possible to administer the DNA methylation inhibitor prior to the administering an HDAC inhibitor to obtain superior results in reestablishing gene expression through synergistic effect. In parallel studies of cancer treatment, induction of tumor suppressors was enhanced by the treatment of a DNA methylation inhibitor followed by the treatment of an HDAC inhibitor.

In one embodiment, a DNA methylation inhibitor is a cytidine analog or derivative thereof. Examples of the cytidine analogs or derivatives include, but art not limited, to 5-azacytidine and 5-aza-2′-deoxycytidine. In a preferred variation of this embodiment, the DNA methylation inhibitor is 5-aza-2′-deoxycytidine (5-aza-CdR or decitabine).

According to this embodiment, the histone deacetylase inhibitor is selected from the group consisting of hydroxamic acids, cyclic peptides, benzamides, short-chain fatty acids, and depudecin.

Examples of hydroxamic acids and hydroxamic acid derivatives include, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. Examples of cyclic peptides include, but are not limited to, trapoxin A, apicidin and depsipeptide. Examples of benzamides include but are not limited to MS-27-275. Examaples of short-chain fatty acids include but are not limited to butyrates (e.g., butyric acid and phenylbutyrate (PB)).

The compositions, kits and methods of the present invention may be used to treat and/or prevent a wide variety of neurological disorders. Examples of such neurological disorders include, but are not limited to, Aarskog syndrome, Alzheimer's disease, amyotrophic lateral sclerosis (Lou Gehrig's disease), aphasia, Bell's Palsy, Creutzfeldt-Jakob disease, cerebrovascular disease, charcot-Marie-Tooth Disease, Comelia de Lange syndrome, dementia, dentatorubral-pallidoluysian atrophy, encephalitis, epilepsy and other severe seizure disorders, essential tremor, fragile X syndrome, fibromylagia, headache, hypomelanosis of Ito, Joubert syndrome, Kennedy's disease, Machado-Joseph's diseases, migraines, Moebius syndrome, myotonic dystrophy, neuromuscular disorders (e.g., Guillain-Barre and muscular dystrophy), neuro-oncology disorders (e.g., neurofibromatosis), neuro-immunological disorders (e.g., multiple sclerosis), pain, pediatric neurology (e.g., autism and dyslexia), prion disease, neuro-otology disorders (e.g., Meniere's disease), Parkinson's disease and movement disorders, Phenylketonuria, Pick's disease, progressive supranuclear palsy, Rubinstein-Taybi syndrome, sleep disorders, spinocerebellar ataxia I (SCA1), Smith-Lemli-Opitz syndrome, Sotos syndrome, spinal bulbar atrophy, type 1 dominant cerebellar ataxia, Tourette syndrome, tuberous sclerosis complex, William's syndrome, as well as injury or trauma to the nervous system.

In regard to the kits of the present invention, the kits may comprise a DNA methylation inhibitor such as decitabine in combination with one or more histone deacetylase inhibitors. In one particular embodiment, the DNA methylation inhibitor is decitabine and the histone deacetylase inhibitor is depsipeptide.

In regard to the methods of the present invention, the method may comprise administering to a patient susceptible to or suffering from a neurological disorder a therapeutically effective amount of a DNA methylation inhibitor such as decitabine and 5-azacytidine and a therapeutically effective amount of a histone deacetylase inhibitor. When a combination treatment is used, a synergistic effect would require a reduced amount of each composition administered. The DNA methylation inhibitor and the histone deacetylase inhibitor may be delivered separately or in combination. In a preferred embodiment, the DNA methylation inhibitor is administered prior to administering the histone deacetylase inhibitor.

The DNA methylation inhibitor and the histone deacetylase inhibitor may be delivered by various routes of administration. For example, they may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms. In a preferred embodiment, the DNA methylation inhibitor is administered intravenously or subcutaneously, and the histone deacetylase inhibitor is administered intravenously. In another preferred embodiment, the DNA methylation inhibitor and the HDAC inhibitor are administered in an alternating sequence (e.g., a 3 day treatment of a DNA methylation inhibitor followed by a one day treatment of an HDAC inhibitor). These recurring treatments may be repeated multiple times or until symptoms subside.

The DNA methylation inhibitor (e.g., decitabine and 5-azacytidine) may be administered to the patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/m², optionally 1-20 mg/m², or optionally 5-30 mg/m².

In one embodiment, the DNA methylation inhibitor (e.g., decitabine and 5-azacytidine) is administered intravenously to the patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/M², optionally 1-20 mg/M², or optionally 5-30 mg/m².

In another embodiment, decitabine is administered into the patient via an 1-24 hour i.v. infusion for 3-5 days per treatment cycle at a dose preferably ranging from 1-100 mg/m² per day, or more preferably at a dose ranging from 2-50 mg/M², or more preferably at a dose ranging from 5-20 mg/M². The preferred dosage below 50 mg/m² for decitabine is considered to be much lower than that used in conventional chemotherapy of decitabine for leukemia.

In another embodiment, the DNA methylation inhibitor (e.g., decitabine and 5-azacytidine) is administered subcutaneously to the patient at a dose of 0.1-1000 mg/m², optionally 1-200 mg/m², optionally 1-150 mg/M², optionally 1-100 mg/m², optionally 1-75 mg/m², optionally 1-50 mg/M², optionally 1-40 mg/m², optionally 1-30 mg/M², optionally 1-20 mg/m², or optionally 5-30 mg/M².

In another embodiment, the histone deacetylase inhibitor is depsipeptide. According to this embodiment, depsipeptide is administered to a patient by continuous i.v. infusion for at least 4 hours at a dose preferably ranging from 2-100 mg/m², more preferably at a dose ranging from 5-50 mg/M², or more preferably at a dose ranging from 5-15 mg/M². This treatment cycle may be repeated several times a month.

The formulation for the continuous i.v. infusion of depsipeptide may be formed by resuspending up to 5 mg/ml of depsipeptide in an ethanol based. The suspension is then further diluted in normal saline for i.v. administration.

In yet another embodiment, the histone deacetylase inhibitor is phenylbutyrate (PB). According to this embodiment, PB is administered to a patient by continuous i.v. infusion for 2 to 3 weeks at a dose preferably ranging from 100-2000 mg/m², more preferably at a dose ranging from 250-1000 mg/m², or more preferably at a dose ranging from 500-800 mg/m².

### BRIEF DESCRIPTION OF THE FIGURES

FIG. 1 illustrates chemical structures for 5-azacytidine and 5-aza-2′-deoxycytidine.

FIG. 2 illustrates chemical structures for some histone deacetylase inhibitors.

### DETAILED DESCRIPTION OF THE INVENTION

A. Neurological Disorders in General

The present invention provides new and improved compositions, kits, and methods for preventing and/or treating patients with neurological disorders using a DNA methylation inhibitor and/or a histone deacetylase inhibitor. By administering the DNA methylation inhibitor to the patient, transcriptional repression of genes that associated with prevention of the neurological disorders or restoration of normal functions can be effectively inhibited through hypomethylation. In addition, coupled with administration of a histone deacetylase inhibitor, the transcriptional repression can be further alleviated through inhibition of deacetylation of histones. Thus, by inhibiting these harmful biochemical modifications of genes (methylation) and histones (deacetylation), transcription of the genes that have been silenced or suppressed can be restored, leading to gain of function of those genes.

Neurological disorders include, for example, Aarskog syndrome, Alzheimer's disease, amyotrophic lateral sclerosis (Lou Gehrig's disease), aphasia, Bell's Palsy, Creutzfeldt-Jakob disease, cerebrovascular disease, charcot-Marie-Tooth Disease, Comelia de Lange syndrome, dementia, dentatorubral-pallidoluysian atrophy, encephalitis, epilepsy and other severe seizure disorders, essential tremor, fragile X syndrome, fibromylagia, headache, hypomelanosis of Ito, Joubert syndrome, Kennedy's disease, Machado-Joseph's diseases, migraines, Moebius syndrome, myotonic dystrophy, neuromuscular disorders (e.g., Guillain-Barre and muscular dystrophy), neuro-oncology disorders (e.g., neurofibromatosis), neuro-immunological disorders (e.g., multiple sclerosis), pain, pediatric neurology (e.g., autism and dyslexia), prion disease, neuro-otology disorders (e.g., Meniere's disease), Parkinson's disease and movement disorders, Phenylketonuria, Pick's disease, progressive supranuclear palsy, Rubinstein-Taybi syndrome, sleep disorders, spinocerebellar ataxia I (SCA1), Smith-Lemli-Opitz syndrome, Sotos syndrome, spinal bulbar atrophy, type 1 dominant cerebellar ataxia, Tourette syndrome, tuberous sclerosis complex, William's syndrome, or any other injury or trauma to the nervous system.

One example of a progressive degenerative neurological disorder is amyotrophic lateral sclerosis (ALS), also known as “Lou Gehrig's disease.” Approximately 30,000 individuals in the United States have ALS, which attacks motor neurons in the brain and spinal cord. When the motor neurons die, the ability of the brain to initiate and control muscle movement is lost. This can lead to paralysis and also death. As such, the life expectancy of ALS patients is typically 3 to 5 years after diagnosis. Early symptoms of ALS include increasing muscle weakness, especially involving the arms and legs, speech, swallowing and breathing. Later as muscles no longer receive messages from the motor neurons, they begin to atrophy.

A number of inherited conditions increase the risk factor for ALS. These conditions include an inherited genetic defect on chromosome 21 in the coding region for enzyme superoxide dismutase (SOD1). SOD1 codes an antioxidant that protects motor neurons from free radical damage. More than 60 different mutations that cause SOD1 to lose its antioxidant properties have been discovered.

Two additional gene loci for recessive ALS have also been discovered on chromosomes 2 and 15. In particular, a mutation on chromosome 2q33 (a GTPase regulator encoding genes) has been associated with both a rare, slowly progressive, early-onset form of the disease called juvenile ALS, or ALS2, as well as to juvenile primary lateral sclerosis (JPLS).

Other genes associated with sporadic ALS include the neurofilament heave (NF-H) gene as well as the androgen receptor gene in X-linked bulbospinal neuronopathy (SBMA or Kennedy's disease). Yang et al. (2001) Nature Genetics October Vol. 29, 160-165; and Hadano et al. (2001) Nature Genetics October Vol. 29, 166-173. The NF-H protein includes a unique phosphorylation domain of multiple lysine-serine-proline (KSP) repeats located in the side-arms, appearing to modulate the spacing between neurofilaments. One form of NF-H sequence contains 43 KSP repeats. However, an NF-H allelic variant containing 44 KSP repeats has been identified. The distribution of the 43 and 44 NF-H allelic variants has been examined in DNA samples from 148 control individuals and 273 non-related individuals with sporadic ALS. The allelic distribution between the two groups varies significantly. Moreover, in 3 ALS patients, mutations have been found in the phosphorviation domain of NF-H. One ALS patient has a 102 bp deletion, which includes 5 KSP repeats, while two other MND/ALS patients have a mutant NF-H allele with Q3 bp deletion including a lysine residue. These mutations may alter the cross-linking properties of NF-H, therefore resulting in an impairment of neurofilament transport.

Another mutation associated with ALS is in the coding region of the glutamate transporter protein EAAT2. EAAT2 is normally responsible for deactivating and recycling glutamate; a chemical that acts as a messenger between neurons though at high levels can be toxic. Cells called astrocytes use the glutamate transporter EAAT2 to absorb excess glutamate and protect neurons. However, it appears that ALS patients have little or no EAAT2 in certain areas of the brain and spinal cord, which results in the accumulation of glutamate causing damage to the motor neurons. Thus, it is proposed that ALS may be caused by a loss of expression of EAAT2.

Mutations in EAAT2 appear to cause more than half of all non-inherited or sporadic cases of the ALS, which comprise approximately 95% of all cases (roughly 30,000 in the United States). EAAT2 becomes mutated during the process of transcribing the EAAT2 from DNA to RNA in which some introns are kept while at least one exon is discarded. The mutated EAAT2 RNA is generally found only in those locations where motor nerve cells are dying (e.g., in the spine and muscle control areas in the brain).

Alzheimer's disease (AD) is another example of a degenerative neurological disorder. AD is characterized by memory loss, language deterioration, impaired visuospatial skills, poor judgment, indifferent attitude, but preserved motor function. It is believed that up to 4 million Americans suffer from AD. AD is caused by loss of nerve cells in areas of the brain that are vital to memory and other mental abilities and usually afflicts people over the age of 60. AD can be classified as early-onset (before age 65) and late-onset (after age 65). It can also be classified as inherited (familial) or sporadic. Familial AD cases represent only 5% of all AD cases.

In families where multiple members have early-onset Alzheimer's, scientists have isolated mutations in three genes that are autosomal dominant to Alzheimer's disease: (i) the amyloid protein precursor (APP) gene on chromosome 21, (ii) the presenilin 1 (PS1) gene on chromosome 14, and (iii) the presenilin 2 (PS2) gene on chromosome 1.

APP is an integral membrane protein occurring in different isoforms. Proteolytic cleaves of APP results in the generation of amyloid-beta proteins (Aβ), of which there are two versions —a shorter one that is harmless and a longer, stickier one that clusters into the characteristic amyloid plaques found in the brains of people with Alzheimer's. Aβ is continuously produced in the brain, and deposition of Aβ in the brain occurs during normal aging but is accelerated in AD patients. It has been suggested that decreased clearance of Aβ from the brain and cerebrospinal fluid is the main cause of Aβ accumulation in sporadic AD. Cell-surface receptors such as the receptor for advanced glycation end products (RAGE), scavenger receptor type A (SR-A), LDL receptor-related protein-1, and LRP-2 bind Aβ at low nanomolar concentrations and may be required for clearance of Aβ across the blood brain barrier. See Shibata M., t al., J. Clin. Invest., (2000) 106(12):1489-1499. Moreover, it has been shown that mutations clusters around the sites of proteolytic cleavage of APP result in an outcome that a more depositable fragment of Aβ is released.

PS1 is a transmembrane protein. Mutations in PS1 result in a more virulent form of Alzheimer's that occurs in people in their 40s and progress quickly. Evidence shows that PS1 mutations may increase the production of Aβ42. While the onset of PS1 encoded AD are typically not modulated by apolipoprotein E genotype, it appears that mutated PS1 leads to disease by causing loss of function of the wild type allele. See Hardy, J. “The Genetic Causes of Neurodegenerative Disease,” J of Alzheimer 's Disease (2001) 3:109-116. This is in agreement with ex PS2 is another transmembrane protein. Genetic mutations in PS2 are rare but may lead to later onset of Alzheimer's relative to PS1 mutations. However, it is suggested that PS2 does not have a large effect on APP processing and that it's function can be substituted for by PS1. Id.

Other genes that show aberrant expression in AD patients include GAP-43, metallothionein (MT)-3, and muscarinic (M)-4 receptor. GAP-43 is a growth-associated phosphoprotein expressed at high levels in neurons during development, axonal regeneration, and neuritic sprouting. Downregulation and aberrant neuronal GAP-43 gene expression appears to correlate with the onset of widespread synaptic disconnection and dementia in AD. See de la Monte SM, et al., Am J Pathol. 1995 October;147(4):934-46. MT-3 is a brain-specific isomer of MT growth inhibitory factors whose expression is significantly reduced in patients with Alzheimer's disease. See Dajun Deng et al., HGM2002 Abstracts, Poster 107. The molecular mechanism of MT-3 downregulation is unknown, but treatment with 5-azacytidine can cause re-expression of MT-3 in brain tissue. Id. Furthermore, it has been shown that muscarinic (M)-4 receptor subtype is selectively reduced in the hippocampus of Alzheimer's patients. See Mulugeta E, Brain Res. (2003) Jan 17;960(1-2):259-62.

The fragile X syndrome is a common form of inherited neurological disorders characterized by mental retardation and developmental disability. This condition afflicts approximately 1 in 1250 males and 1 in 2000 females. As the name implies, fragile X is an X chromosome-linked condition. The fragile X phenotype is characterized by a visible constriction near the end of the X chromosome, at locus q27.3, and there is a tendency for the tip of the X-chromosome to break off under certain conditions in tissue culture. These tissue culture procedures form the basis of the assay most commonly used for fragile X at present.

The pattern of inheritance of this condition is atypical of that associated with X-linked conditions. Typically, there is a 50% probability that the son of a woman who carries an X-linked genetic defect will be afflicted by the defect. Additionally, all males who carry the abnormal gene are afflicted by the X-linked condition in the typical pattern. Furthermore, since females have two X chromosomes, they normally do not suffer the effects of a single damaged X chromosome.

In fragile X, however, some carrier males are phenotypically normal. Moreover, about one third of the females who inherit the fragile X chromosome are afflicted. The incidence of carrier males in different generations of a family varies. Daughters of carrier males are generally non-expressing carriers, but may have afflicted sons. Furthermore, afflicted daughters occur more frequently among the offspring of carrier mothers than among the offspring of carrier fathers. See Brown, The Fragile X: Progress toward Solving the Puzzle, Am. J. Human Genet. 47 175-80, 1990. This and all other references, patents and patent applications are incorporated herein by reference for all purposes.

Researchers have identified the genomic region associated with this condition. (Oberle, et al., “Instability of a 550-Base Pair DNA Segment and Abnormal Methylation in Fragile X Syndrome”, Science 252 1097-1102, 1991; Kremer, et al., “Mapping of DNA Instability at the Fragile X to a Trinucleotide Repeat Sequence p(CCG)n”, Science 252 1711-14, 1991; and Bell, et al., “Physical Mapping across the Fragile X Hypermethylation and Clinical Expression of the Fragile X Syndrome”, Cell 64 861-66, 1991). Additionally, researchers have sequenced a partial cDNA clone derived from this region, called FMR-1. (Verkerk, et al., “Identification of a Gene (FMR-1) Containing a CGG Repeat Coincident with a Breakpoint Cluster Region Exhibiting Length Variation in Fragile X Syndrome”, Cell 65 905-14, 1991). These studies provide an explanation for the atypical pattern of inheritance of fragile X. The mutation that ultimately results in the fragile X phenotype occurs in stages. In the early stages, the gene is not fully defective, rather there is a “pre-mutation” of the gene. Carriers of the premutation have a normal phenotype. A further mutation occurs in carrier females that produce the phenotype in their offspring.

The coding sequence for FMR-1 contains a variable number of CGG repeats. Individuals who are not carriers have approximately 30 CGG repeats in their FMR-1. Carriers, however, have between 50 and 200 CGG repeats. This amplification of the FMR-1 CGG sequence is the pre-mutation. Afflicted individuals have even more CGG repeats. As many as several thousand CGG repeats have been observed in afflicted individuals. (Oberle, et al., 1991, supra).

However, most affected individuals do not express the FMR-1 mRNA (Pieretti, et al., Absence of Expression of the FMR-1 Gene in Fragile X Syndrome, Cell 66 1-201991). A CpG island, located upstream of the CGG repeat region, is methylated when the number of CGG repeats is above a threshold of about 200 copies (Oberle, et al., 1991; Kremer, et al., 1991, Bell, et al., 1991, supra). This methylation inactivates the gene.

It has been recognized that fragile X syndrome is caused by loss of expression of FMRP, a protein proposed to act as a regulator of mRNA translation which promotes synaptic maturation and function. FMRP has been found to associate with the RNP complex that mediates post-transcriptional silencing by RNA. See review by Carthew RW. “RNA interference: the fragile X syndrome connection” Curr. Biol. 2002 December 23;12(24):R852-4.

There is still no medication for fragile X syndrome which acts directly on the genetic mechanisms or on the immediate result of the genetic defect. However, behavioral and cognitive manifestations can be approached from both the psychological/educational and pharmacological sides. Currently there are drugs that can improve important symptoms of the fragile X syndrome, behavioral disorders, hyperactivity, attention deficit, obsessive disorders and anxiety, including central nervous system stimulants, clonidine, folic acid, serotonin reuptake inhibitors, and atypical antipsychotics (Artigas Pallares J, Brun Gasca C. “Medical treatment of fragile X syndrome” Rev Neurol 2001 October 1;33 Suppl 1:S41-50).

Spinocerebellar ataxia type 1 (SCA1) is another degenerative neurological disorder caused by an error in the gene that codes for the protein known as ataxin-1. SCA1 and related diseases are known as polyglutamine diseases because the resulting mutant protein has an unusually long polyglutamine tract. The underlying mechanism for this disorder involves a piece of DNA consisting of CAG repeats that becomes amplified, leading to a protein product that contains a pathologically expanded string of glutamine residues. The mutant protein tends to clump inside the nucleus of the cell making it difficult for the neurons to recycle the mutant proteins. SCA1 is characterized by the onset (usually in adulthood) of cerebellar and bulbar dysfunction. This is due to a severe loss of cerebellar Purkinje cells as well as atrophy and degeneration in various other regions of the brain and spinal cord. Experiments in mice show that certain genes become downregulated before the onset of SCA1 while others become upregulated after the onset of SCA1. See Lin X., (2000) Nat. Neurosci. 3, 157-163; Nussbaum R., et al., Nature, (2000) Vol. 3 No. 2:103. Genes that are downregulated include prenylcysteine carboxylmethyltransferase (PCCMT), an enzyme enriched in the endoplasmic reticulum (ER) of the cerebellum that participates in post-translational lipid modification of many proteins, including the G protein RAS. Downregulation of PCCMT was observed one day after the Purkinje-cell specific promoter began to drive expression of the ataxin-1 transgene in nude mice and at least 5-6 weeks before the first manifestations of the disease. Other downregulated genes include type I ER inositol triphosphate receptor (IP3R1), inositol polyphosphate 5-phosphatase (INPP5A), an ER calcium pump (SERCA2), the calcium ion channel TRP3, and the glutamate transporter EAAT4. Three of these genes (PCCMT, SERCA2 and IP3R1) were shown to be downregulated in early-onset of SCA1 human patients as well. Downregulation of these genes occurs approximately 2-3 weeks before the onset of the disease. After the onset, it has been noticed that alphal-antichymotrypsin is upregulated. Downregulation of the genes above may promote excitotoxicity, a well-characterized phenomenon in which neurons die by apoptosis due to over-excitation of glutamate receptors and a consequent increase in cytoplasmic calcium.

Parkinson's disease (PD) is a progressive degenerative neurological disorder that affects nearly 1,000,000 Americans. PD is characterized by deposits in the brain called Lewy bodies and is caused by severe shortage of dopamine, a neurotransmitter that acts as a chemical messenger between nerve cells. In the brain, dopamine levels are mediated, in part, by the expression of dopamine receptors, D1 and D2. Gerfen, C. R., Science (1990) 7:250(4986):1429-32. Reduced levels of dopamine may result in symptoms such as rigidity or stiffness in the muscles, tremor, bradykinesia or slowness of movement, poor balance, decreased or non-existent arm swing, difficulty in negotiating turns and sudden freezing spells causing an inability to take the next step.

Several genes (e.g., UCH-LI, alpha-synuclein, parkin and Dj-1) and their corresponding protein products are known for being involved in Parkinson's disease. Some of these genes are related to the ubiquitin proteasome pathway (UPP), which degrades proteins. The UPP is composed of ubiquitin, a tiny molecule that binds damaged protein and carries it to a proteasome where the protein is degraded. Abnormal proteasome may be associated with an onset of PD or other neurodegenerative disease. Bence N F, Sampat R M, Kopito R R. Impairment of the ubiquitin-proteasome system by protein aggregation. Science. 2001 May 25;292(5521); 1552-5.

UCH-L1 (ubiquitin carboxy-terminal hydrolase L1), another protein associated with UPP, comprises 1% -2% of all the proteins in the brain and can also be found in Lewy bodies. UCH-L1 is a de-ubiquitinating enzyme that hydrolyzes bonds between ubiquitin molecules that are attached to other proteins, to create monomeric (single) ubiquitin molecule. A missense mutation in UCH-L1 occurs in an autosomal-dominant form of PD, resulting in the replacement of an isoleucine by a methionine. This change causes a decrease in the hydrolytic activity of UCH-L1 and a decrease in available ubiquitin, which leads to a buildup of toxic proteins in neurons. UCH-L1 is linked to familial PD. Solano, S M, Ann Neurol. (2000) 47(2):201-10.

Other genes associated with PD include pakin, alpha-synuclein, Dj-1 and tau. Parkin is a 465 amino acid protein and an E3 ligase encoded by the parkin gene on chromosome 6. Parkin has been associated with early and late-onset PD. Lewy bodies do not appear in the brains of patients with Parkinson's disease resulting from parkin mutations.

Known substrates that parkin ubiquitinates include Pael-R, modified alpha-synuclein, CDCrel-1 and Synphilin-1. Pael-R (parkin-associated endothelin-receptor-like receptor) is possibly a G protein-coupled transmembrane protein. When Pael-R unfolds it becomes insoluble and accumulates in the ER. If it is ubiquitinated by parkin, it degrades by the UPP; otherwise, it leads to cell death. Modified alpha-synuclein is a 22-kDa glycosylated form of alpha-synuclein and is ubiquitinated by parkin. If parkin is mutated, modified alpha-synuclein accumulates in the cell and may result in cell death. CDCrel-1 (cell-division-control-related protein 1) is a septin GTPase and may regulate synaptic vesicle release. Synphilin-1 interacts with alpha-synuclein and is found in Lewy bodies. Parkin mutations may result in the death of dopamine neurons in Parkinson's disease, as the abnormal buildup of parkin's substrates may be toxic to the cell. When normal parkin is present, such proteins would be destroyed.

Alpha-synuclein is a 140 amino acid protein that is abundant in the brain and has a tendency to form insoluble aggregates particularly in its mutated form. The alpha-synuclein gene is located on chromosome 4. It has been demonstrated that alpha-synuclein forms a tight 2:1 complex with histones and that the fibrillation rate of alpha-synuclein is dramatically accelerated in the presence of histones in vitro. See Goers J, et al. “Nuclear Localization of alpha-synuclein and Its Interaction with Histones,” Biochemistry (2003) Jul. 22;42(28):8465-8471. Furthermore, alpha-synuclein co-localizes with histones in the nuclei of nigral neurons from mice exposed to a toxic insult. Id. Interestingly, alpha-synuclein mutations are also linked to Alzheimer disease.

The gene Dj-1 is also linked to Parkinson's disease. See Vincenzo B., Science Jan. 10 2003: 256-259. Published online Nov. 21, 2002. Mutations in Dj-1 are associated with a form of Parkinson's disease known as PARK7, an autosomal recessive early onset form of the disease. The mutation in Dj-7 results in a change from the amino acid leucine to proline at amino acid position 166 in the protein.

Furthermore, late-onset of PD is associated with chromosome 17 tau gene. Tau is a component of neurofibrillary tangles, a specific brain abnormality found in other neurodegenerative disorders. The familial link to chromosome 9 was found primarily in patients who do not respond to levodopa (a precursor of dopamine and a common treatment for PD). The marker for familial PD is located near another gene that is altered in idiopathic torsion dystonia. This suggests a possible relationship between PD and dystonia.

Another example of a neurological disorder is tuberous sclerosis complex (TSC). TSC is an autistic disorder observed in some patients. This inherited condition may lead to seizures, mental retardation, and unusual skin conditions. There are two different genes that are associated with TSC, one on chromosome 9 (TSC1) and the other on chromosome 16 (TSC2). An individual with this disorder will have a mutation in only two one of these genes. When an individual has TSC, there is a 50% chance that his or her offspring, regardless of sex, will inherit the same TSC mutation. Currently TSC is most easily diagnosed by a physical examination that includes a Wood's lamp examination of the skin.

Neurofibromatosis Type 1 (NF1) is an inherited neurological disorder that may lead to unusual skin findings, tumors in the central nervous system and learning disabilities. NF1 is caused by mutations in a gene on chromosome 17. Like TSC, if an individual has NF1, there is a 50% chance that his or her offspring will inherit a NF1 gene mutation, and therefore be likely to develop symptoms of NF1. Because symptoms of NF1 may vary even among family members, sometimes individuals may not be aware that they have a NF1 gene mutation. Currently NF1 is most easily diagnosed by a physical examination, but DNA testing to confirm the diagnosis is possible in many instances.

X-linked spinal and bulbar muscular atrophy, or Kennedy's disease, is a recessive, adult-onset form of lower motor neuron degeneration also associated with signs of androgen insensitivity. The androgen receptor gene has been mapped to chromosome Xq11-12, where linkage studies have localized the SBMA gene defect. The first exon of the gene contains a polymorphic CAG repeat coding a polyglutamine stretch. The number of the CAG repeats normally varies in the population between 15 and 33. However, in patients with Kennedy's disease, the number of repeats varies from 40 to 52. Thus, there is an absolute association of the larger polyglutamine stretch with the disease phenotype and, furthermore, the number of the repeats correlates with the severity of the disease. This is the only known mutation of the androgen receptor gene associated with motor neuron degeneration. Functional studies of the receptor carrying the expanded polyglutamine stretch show that the mutated protein exhibits reduced transcriptional competence.

B. Reestablishing Gene Expression

According to the present invention, aberrant transcriptional silencing of a number of genes, such as neurotransmitters, neurotransmitter receptors and transcription factors (e.g., TATA-binding proteins and CREB-binding protein), is directly related to pathogenesis of neurological disorders. Such neurological disorders include, but are not limited to, Aarskog syndrome, Alzheimer's disease, amyotrophic lateral sclerosis (Lou Gehrig's disease), aphasia, Bell's Palsy, Creutzfeldt-Jakob disease, cerebrovascular disease, charcot-Marie-Tooth Disease, Cornelia de Lange syndrome, dementia, dentatorubral-pallidoluysian atrophy, encephalitis, epilepsy and other severe seizure disorders, essential tremor, fragile X syndrome, fibromylagia, headache, hypomelanosis of Ito, Joubert syndrome, Kennedy's disease, Machado-Joseph's diseases, migraines, Moebius syndrome, myotonic dystrophy, neuromuscular disorders (e.g., Guillain-Barre and muscular dystrophy), neuro-oncology disorders (e.g., neurofibromatosis), neuro-immunological disorders (e.g., multiple sclerosis), pain, pediatric neurology (e.g., autism and dyslexia), prion disease, neuro-otology disorders (e.g., Meniere's disease), Parkinson's disease and movement disorders, Phenylketonuria, Pick's disease, progressive supranuclear palsy, Rubinstein-Taybi syndrome, sleep disorders, spinocerebellar ataxia I (SCA1), Smith-Lemli-Opitz syndrome, Sotos syndrome, spinal bulbar atrophy, type 1 dominant cerebellar ataxia, Tourette syndrome, tuberous sclerosis complex, William's syndrome, as well as injury or trauma to the nervous system.

Methylation of cytosine residues in DNA and removal of acetyl groups from histones are the two primary mechanisms for gene silencing. Due to methylation and/or histone deacetylation of neurotransmission-related genes, expression of these genes is suppressed or completely silenced. Meanwhile, expression of these genes is required for normal neuronal functions and neurotransmission. Inaction of these genes in the effected cells can lead to neurodegeneration, which eventually results diseases or conditions such as those disclosed herein.

For example, for fragile X syndrome, hypermethylation of the DNA at the FMR-1 locus is responsible for variable phenotypic expression of the fragile X syndrome. Such hypermethylation at several different sites on the promoter region of the FMR-1 gene shuts down the expression of gene, leading to loss of the function of gene product, FMRP, a protein that acts as a regulator of mRNA translation and promotes synaptic maturation and function. It would be useful to upregulate or reestablish transcription of FMRP.

In another example, a vast majority of patients suffering from spontaneous ALS express little or no EAAT2, a glutamate transporter. As a result of this underexpression of EAAT2, glutamate accumulates in brain regions that control motor functions. An excess amount of glutamate causes motor neuron degeneration. Therefore, it would be useful to upregulate or reestablish transcription of EAAT2.

Other genes that may be reestablished include, for example, SOD-1 for ALS; GAP-43, MT-3 and M(4) receptor for Alzheimer's disease; and UCH-L1, parkin, alpha-synuclein, Dj-1, and tau for Parkinson's disease.

The present invention offers an effective method for reactivating the genes required for normal neuronal functions and neurotransmission whose expression has been suppressed by DNA methylation. The method, in general, comprises administering to a patient with a neurological disorder a therapeutically effective amount of a DNA methylation inhibitor. The method can also be utilized to prevent the onset of neurological disorders.

According to the present invention, the DNA methylation inhibitor inhibits methylation of DNA for the genes, especially in the regulatory region, thus resulting in activation of transcription of the gene. The DNA methylation inhibitor is preferably a DNA methyltransferase inhibitor.

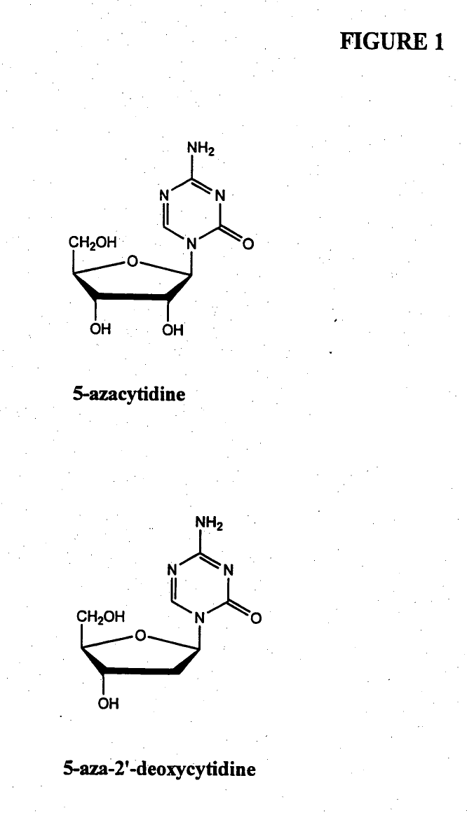

In one embodiment, the DNA methylation inhibitor is a cytidine analog or derivative. Examples of cytidine analogs or derivatives include, but art not limited to, 5-azacytidine and 5-aza-2′-deoxycytidine. In a preferred variation of this embodiment, the DNA methylation inhibitor is 5-aza-2′-deoxycytidine (5-aza-CdR or decitabine). Chemical structures for 5-azacytidine and 5-aza-2′-deoxycytidine are shown in FIG. 1.

Decitabine, 5-aza-2′-deoxycytidine, is an antagonist of its related natural nucleoside, deoxycytidine. The only structural difference between these two compounds is the presence of a nitrogen at position 5 of the cytosine ring in decitabine as compared to a carbon at this position for deoxycytidine. Two isomeric forms of decitabine can be distinguished. The β-anomer is the active form. The modes of decomposition of decitabine in aqueous solution are (a) conversion of the active b-anomer to the inactive β-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent forming of guanidine compounds (Kissinger and Stemm (1986) J. Chromat. 353:309-318).

Decitabine possesses multiple pharmacological characteristics. At a molecular level, it is capable of specifically inhibiting cell growth at S phase and DNA methylation. At a cellular level, decitabine can induce cell differentiation and exert hematological toxicity. Despite having a short half life in vivo, decitabine has excellent tissue distribution.

The most prominent function of decitabine is its ability to specifically and potently inhibit DNA methylation. As described above for methylation of cytosine in CpG islands as an example, methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substistuting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes can be prevented.

Thus, according to the present invention, the inventors take advantage of the ability of DNA methylation inhibitors, such as decitabine, reactivate the neurologically functional genes silenced by aberrant methylation, such as the FMR-1 gene silenced in the fragile X syndrome, growth inhibitory factor metallothionein-3 silenced in Alzheimer's disease, EAAT2 silenced in ALS disease, and D1, D2, UCH-L1, alpha-synuclein, parkin, Dj-1 silenced in Parkinson's disease. By reducing methylation, expression of FMRP and other genes necessary to maintain normal phenotype can be reactivated, leading to the promotion of synaptic maturation and effective treatment of the neurological disorder.

The present invention also provides a combination therapy for preventing and/or treating neurological disorders. The method comprises administering to a patient susceptible to or with a neurological disorder a therapeutically effective amount of a DNA methylation inhibitor and a histone deacetylase inhibitor.

The DNA methylation inhibitor inhibits methylation of DNA for the neurologically functional genes, especially in the regulatory region, thus resulting in activation of transcription of the gene. Meanwhile, the histone deacetylase inhibitor inhibits deacetylation of the histones in the nucleosomal core of the gene, thus resulting in net increase of the acetylation of histones, which, in turn, activates transcription of the gene. By exploiting these two complementary mechanisms, the combination therapy of the present invention may reestablish gene transcription more effectively and, ideally, in a synergistic manner. A combination therapy having synergistic effects should require a less amount of each inhibitor than it being used alone, thus reducing potential side effects associated systemic administration of high dosages of the inhibitors.

The DNA of all chromosomes is packaged into a compact structure with the aid of specialized proteins. The DNA-binding proteins in eucaryotes are divided into tow general classes: the histones and the nonhistone chromosomal proteins. The complex of both classes of protein with the nuclear DNA of eucaryotic cells is known as chromatin. Histones are unique to eucaryotes and the principal structural proteins of eucaryotic chromosomes. They are present in such enormous quantities that their total mass in chromatin is about equal to that of the DNA.

Up until now there are five types of histones identified in chromatin: H1, H2A, H2B, H3, and H4. These five types of histones fall into two main groups: the nucleosomal histones and the H1 histones. The nucleosomal histones (H2A, H2B, H3, and H4) are small proteins (1-2-105 amino acids) responsible for coiling the DNA into nucleosomes. The H1 histones are larger (containing about 220 amino acids). They occur in chromatin in about half the amount of the other types of histones and appear to lie on the outer portion of the nucleosome.

Histones play a crucial part in packing of chromosomal DNA and activation of genes within. Histones pack the very long helix of DNA in each chromosome in an orderly way into a nucleus only a few micro meters in diameters. The role of histones in DNA folding is important in that the manner in which a region of the genome is packaged into chromatin in a particular cell influences the activity of the genes the region contains.

Chromatin structure of transcribed genes is less decondensed than that of the untranscribed or silenced genes. Studies have shown that transcriptionally active chromatin is biochemically distinct from that of the inactive chromatin. The analysis of the chromosomal proteins in the active chromatin suggested the following biophysical and biochemical characteristics: 1) Histone H1 seems to be less tightly bound to at least some active chromatin; 2) the four nucleosomal histones appear to be unusually highly acetylated when compared with the same histones in inactive chromatin; and 3) the nucleosomal histone H2B in active chromatin appears to be less phosphorylated than it is in inactive chromatin. These changes in chromatin features play an important part in uncoiling the chromatin of active genes, helping to make the DNA available as a template for RNA synthesis during transcription of the gene.

In particular, acetylation and deacetylation of histone plays important roles in regulation of gene expression. It has been demonstrated that chromatin fractions enriched in actively transcribed genes are also enriched in highly acetylated core histones, whereas silent genes are associated with nucleosomes with a low level of acetylation. Kouzarides (1999) Curr. Opin Genet Dev. 9:40-48. Since histones have a very high proportion of positively charged amino acids (lysine and arginine): the positive charge helps the histones bind tightly to DNA which is highly negatively charged, regardless of its nucleotide sequence. Acetylation of histones, particularly in e-amino group of lysine, neutralizes the charge of the histones and generate a more open DNA conformation. Such an open conformation of chromatin DNA provides access to transcription factors and the transcription machinery, which in turn promotes expression of the corresponding genes. Conversely, deacetylation of histones restores positive charge to the amino acids and results in tighter binding of histones to the negatively charged phosphate backbone of DNA. Such a condensed chromatin DNA conformation is relatively inaccessible to the transcription machinery and thus the genes in the condensed area are not expressed, i.e. silenced.

The amount of acetylation on the histones is controlled by the opposing activities of two types of enzymes, histone acetyl transferase (HATs) and histone deacetylases (HDACs). Substrates for these enzymes include e-amino groups of lysine residues located in the amino-terminal tails of the histones H3, H4, H2A, and H2B. These amino acid residues are acetylated by HATs and deacetylated by HDACs. With the removal of the acetyl groups from the histone lysine by HDACs, a positive charge is restored to the lysine residue, thereby condensing the structure of nucleosome and silencing the genes contained within. Thus, to activate these genes silenced by deacetylase of histones, the activity of HDACs should be inhibited. With the inhibition of HDAC, histones are acetylated and the DNA that is tightly wrapped around a deacetylated histone core relaxes. The opening of DNA conformation leads to expression of specific genes.

According to the present invention, a combination therapy with a DNA methylation inhibitor and an HDAC should be particularly useful for treating the neurological disorders herein, especially ALD. In patients with ALS there are elevated levels of glutamate as a result of downregulation of EAAT2. Thus, inhibition of deacetylation by using an HDAC inhibit should synergistically reestablish EAAT2, thereby preventing the onset of the symptoms of ALS or thwarting the onset or progression of the disease.

A combination therapy with a DNA methylation inhibitor and an HDAC should also be particularly useful for treating fragile X syndrome. It is recognized that mutation of the FMR1 gene results in fragile X mental retardation. The most common FMR1 mutation is expansion of a CGG repeat tract at the 5′ end of FMRI, which leads to cytosine methylation and transcriptional silencing. Both DNA methylation and histone deacetylation have been associated with transcriptional inactivity. The methyl cytosine-binding protein MeCP2 binds to histone deacetylases and represses transcription in vivo, suggesting that MeCP2 recruits histone deacetylases to methylated DNA, resulting in histone deacetylation, chromatin condensation and transcriptional silencing. It has been demonstrated that the 5′ end of FMR1 is associated with acetylated histones H3 and H4 in cells from normal individuals, but acetylation is reduced in cells from fragile X patients (Coffee et al. (1999) Nature Genet. 22:98-101). Thus, inhibition of deacetylation by using an HDAC inhibit should synergistically inhibit aberrant transcriptional repression exerted by mutant FMR-1 protein, thereby preventing the onset of the symptoms of fragile X syndrome or thwarting the progression of the disease.

In addition to deacetylation of histones, HDACs may also regulate gene expression by deacetylating transcription factors, such as p53 (a tumor suppressor gene), GATA-1, TFIIE, and TFIIF. Gu and Roeder (1997) Cell 90:595-606 (p53); and Boyes et al. (1998) Nature 396:594-598 (GATA-1). HDACs also participate in cell cycle regulation, for example, by transcription repression which is mediated by RB tumor suppressor proteins recruiting HDACs. Brehm et al. (1998) Nature 391:597-601.

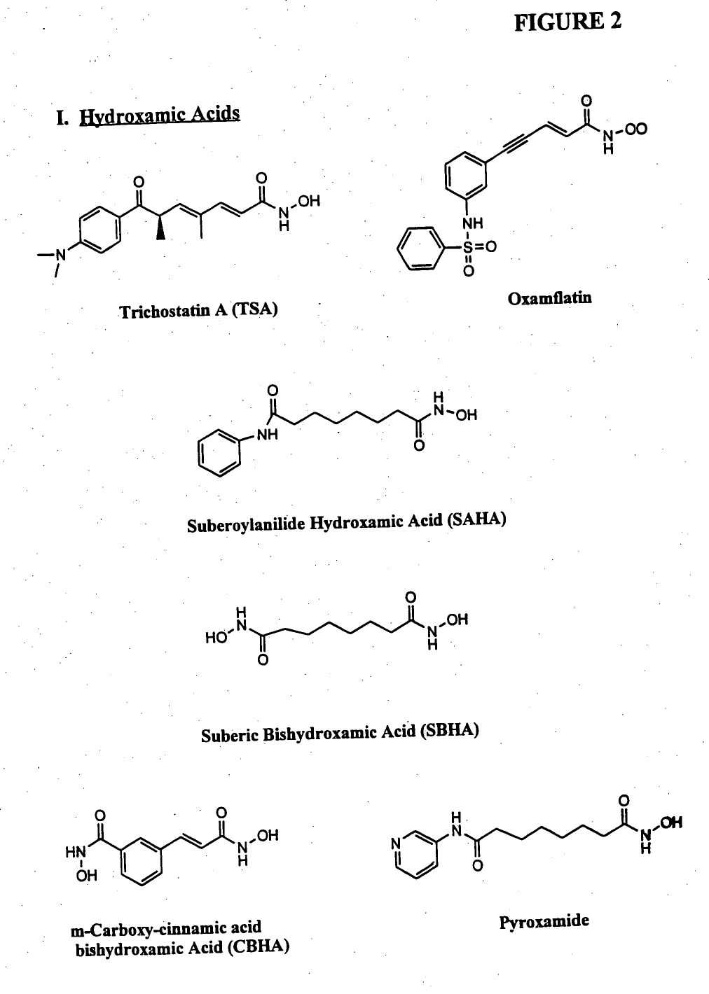

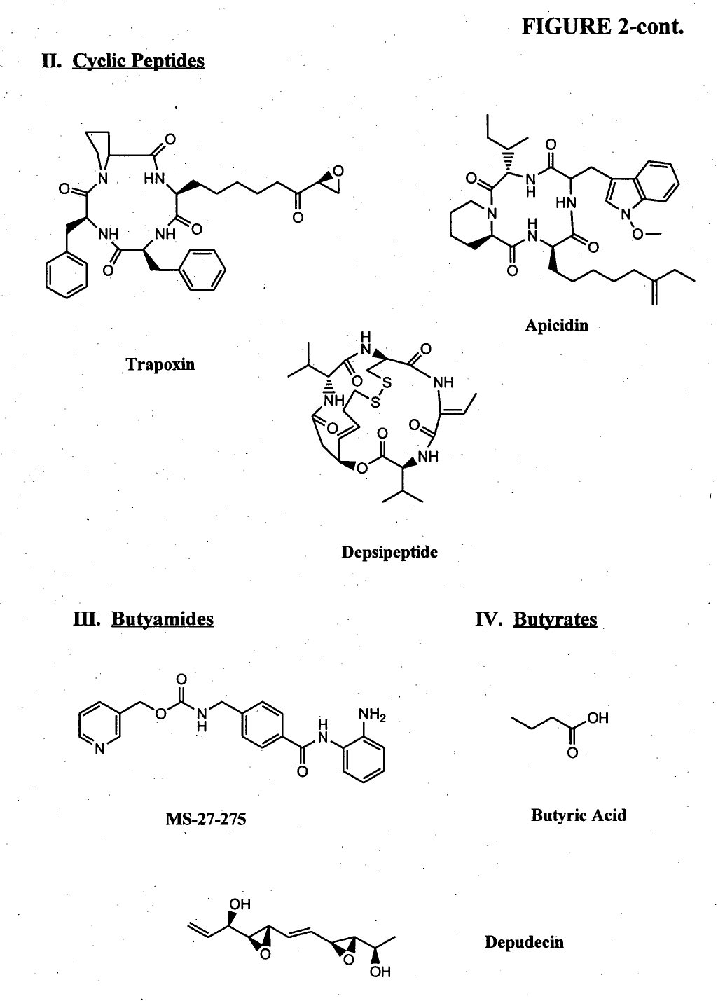

Inhibitors of HDACs include, but are not limited to, the following structural classes: 1) hydroxamic acids, 2) cyclic peptides, 3) benzamides, and 4) short-chain fatty acids. Chemical structures for some of these HDAC inhibitors are shown in FIG. 2. Other forms of HDAC inhibitors include depsipeptide and valproic acid.

According to this embodiment, the histone deacetylase inhibitor is selected from the group consisting hydroxamic acids, cyclic peptides, benzamides, short-chain fatty acids, and depudecin.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and depsipeptide. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activitity and inhibits HDAC activity at nanomolar concentrations. Darkin-Rattray et al. (1996) Proc. Natl. Acad. Sci. USA. 93;13143-13147. Depsipeptide is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597. Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor of the present invention.

C. Delivery

A wide variety of delivery methods and formulations for different delivery methods may be used in administering the DNA methylation inhibitors and the HDAC inhibitors.

The DNA methylation inhibitors and/or the HDAC inhibitors may be administered as compositions that comprise either or both of the therapeutic agents. Such compositions may include, in addition to the inventive combination of therapeutic agents, conventional pharmaceutical excipients, and other conventional, pharmaceutically inactive agents. Additionally, the compositions may include active agents in addition to the inventive combination of therapeutic agents. These additional active agents may include additional compounds according to the invention, or one or more other pharmaceutically active agents. In preferable embodiments, the inventive compositions will contain the active agents, including the inventive combination of therapeutic agents, in an amount effective to treat an indication of interest.

The inventive combination of therapeutic agents and/or compositions may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The DNA methylation inhibitors and the HDAC inhibitors may be administered by a variety of routes, and may be administered, or coadministered in any conventional dosage form. Coadministration in the context of this invention is defined to mean the administration of more than one therapeutic in the course of a coordinated treatment to achieve an improved clinical outcome. Such coadministration may also be coextensive, that is, occurring during overlapping periods of time. For example, the DNA methylation inhibitor may be administered to a patient before, concomitantly, or after the histone deacetylase inhibitor is administered. In a preferred embodiment, the patient may be pretreated with the DNA methylation inhibitor (e.g., decitabine) and then treated with the histone deacetylase inhibitor (e.g., depsipeptide).

Amounts of the inventive combination of therapeutic agents can vary, according to determinations made by one of skill, but preferably are in amounts effective to create a cytotoxic or cytostatic effect at the desired site. Preferably, these total amounts are less than the total amount adding the maximum tolerated dose for each of the DNA methylation inhibitor and the histone deacetylase inhibitor, and more preferably less than the total amount added for individual administration of each of these inhibitors.

For the slow-release dosage form, appropriate release times can vary, but preferably should last from about 1 hour to about 6 months, most preferably from about 1 week to about 4 weeks. Formulations including the inventive combination of therapeutic agents and/or composition can vary, as determinable by one of skill, according to the particular situation, and as generally taught herein.

Decitabine may be supplied as sterile powder for injection, together with buffering salt such as potassium dihydrogen and pH modifier such as sodium hydroxide. This formulation is preferably stored at 2-8° C., which should keep the drug stable for at least 2 years. This powder formulation may be reconstituted with 10 ml of sterile water for injection. This solution may be further diluted with infusion fluid known in the art, such as 0.9% sodium chloride injection, 5% dextrose injection and lactated ringer's injection. It is preferred that the reconstituted and diluted solutions be used within 4-6 hours for delivery of maximum potency. Optionally, the liquid formulation may be infused directly, without prior reconstitution.

The DNA methylation inhibitor (e.g., decitabine and 5-azacytidine) may be co-administered in any conventional form with one or more member selected from the group comprising infusion fluids, therapeutic compounds, nutritious fluids, anti-microbial fluids, buffering and stabilizing agents.

Optionally, the DNA methylation inhibitor (e.g., decitabine and 5-azacytidine) may be formulated in a liquid form by solvating the inventive compound in a non-aqueous solvent such as glycerin, polyethylene glycol, propylene glycol, and ethanol. The pharmaceutical liquid formulations provide the further advantage of being directly administrable, (e.g., without further dilution) and thus can be stored in a stable form until administration. Further, because glycerin can be readily mixed with water, the formulations can be easily and readily further diluted just prior to administration. For example, the pharmaceutical formulations can be diluted with water 180, 60, 40, 30, 20, 10, 5, 2, 1 minute or less before administration to a patient. Other examples of the liquid formulation of decitabine or 5-azacytidine are described in U.S. patent application Ser. No. 10/164,276 which is herein incorporated by reference in its entirety.

The DNA methylation inhibitor (e.g., decitabine and 5-azacytidine) may be administered to the patient at a dose of 0.1-1000 mg/m², optionally 1-200 mg/m², optionally 1-150 mg/m² optionally 1-100 mg/M², optionally 1-75 mg/m², optionally 1-50 mg/m², optionally 1-40 mg/m², optionally 1-30 mg/m², optionally 1-20 mg/m², or optionally 5-30 mg/m².

In one embodiment, the DNA methylation inhibitor (e.g., decitabine and 5-azacytidine) is administered intravenously to the patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/m², optionally 1-20 mg/m², or optionally 5-30 mg/m².

In another embodiment, the DNA methylation inhibitor (e.g., decitabine and 5-azacytidine) is administered subcutaneously to the patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/m², optionally 1-20mg/m², or optionally 5-30 mg/m².

In a preferred embodiment, decitabine is administrated to a patient by injection, such as bolus i.v. injection, continuous i.v. infusion and i.v. infusion over 1 hour. For example, decitabine may administered into the patient via an 1-24 hour i.v. infusion per day for 3-5 days per treatment cycle at a dose preferably ranging from 1-100 mg/m², more preferably ranging from 2-50 mg/m², and most preferably from 5-20 mg/m². The preferred dosage below 50 mg/m² for decitabine is considered to be much lower than that used in conventional chemotherapy for cancer. By using such a low dose of decitabine, transcriptional activity of genes silenced in the cells can be activated to trigger downstream signal transduction for normal neuronal functions. This low dosage, however, should have less systemic cytotoxic effect on normal cells, and thus have less side effects on the patient being treated.

For the histone deacetylase inhibitor, the dosage form depends on the type of compound used as the inhibitor. For example, depsipeptide may be formulated for i.v. infusion.

In one embodiment, depsipeptide is administered to a patient by continuous i.v. infusion for at least 4 hours at a dose preferably ranging from 1-100 mg/m², more preferably at a dose ranging from 2-50 mg/m², and more preferably at a dose ranging from 5-25 mg/m². Treatment with depsipeptide may be repeated numerous times per month, preferably at even intervals (every 3 days, weekly, bi-monthly, etc.). In another embodiment, depsipeptide is administered to a patient by continuous i.v. infusion for at least 4 hours per day for a week at a dose preferably ranging from 2-100 mg/m², more preferably ranging from 5-50 mg/m², and most preferably from 5-15 mg/m². The treatment cycle may be 1 or 2 weeks per month.

In another embodiment, phenylbutyrate (PB) is administered to a patient by continuous i.v. infusion at a dose preferably ranging from 100-2000 mg/m², more preferably at a dose ranging from 250-1000 mg/m², and more preferably at a dose ranging from 500-800 mg/m².

In another embodiment, arginine butyrate is administered to a patient by continuous i.v. infusion at a dose preferably ranging from 100-2000 mg/m², more preferably at a dose ranging from 250-1000 mg/m², and more preferably at a dose ranging from 500-800 mg/m² For example, arginine butyrate may be administered at a dose between 250-1000 mg/m² as a 6-12 hour iv infusion for 4 days every 2 weeks.

In preferred embodiment, depsipeptide is administered after administration of decitabine to the patient. This clinical regimen is designed to enhance efficacy of the combination therapy by sensitizing the neurons through inhibition of methylation.

The inventive combination of therapeutic agents may be used in the form of kits. The arrangement and construction of such kits is conventionally known to one of skill in the art. Such kits may include containers for containing the inventive combination of therapeutic agents and/or compositions, and/or other apparatus for administering the inventive combination of therapeutic agents and/or compositions.

### EXAMPLES

### Example 1

In one example, a patient suffering from ALS is administered decitabine by intravenous injection at a dose rate of 10-50 mg/m² per day for three days. On the fourth day of treatment the patient is administered an HDAC inhibitor such as depsipeptide or Trichostatin A (TSA), which have similar potency. The depsipeptide or TSA is administered at a dose of 5-20 mg/m², preferably in a four-hour infusion. This four-day treatment course can be repeated multiple times or until EAAT2 expression is reestablished in the spine and muscle control regions in the brain.

### Example 2

another example, a healthy patient or patient susceptible to a neurological disorder such as Alzheimer's disease is administered a prophylactic treatment comprising of decitabine. The decitabine is administered by intravenous injection at a dose rate of 5-20 mg/m² per day for 1-4 days. The patient can also be administered an HDAC inhibitor simultaneously or after the decitabine treatment. The HDAC inhibitor can be phenylbutyrate and administered at a dose ranging from 250 to 1000 mg/m². This treatment plan can be repeated multiple times or until expression of the gene of interest (e.g., GAP-43, growth inhibitory factor metallothionein-3, and muscarinic-4 receptor subtype) are reestablished.

### Example 3

In another example, a patient suffering from Parkinson's disease is administered decitabine by subcutaneously at a dose rate of decitabine alone, or in combination with an HDAC inhibitor. Decitabine is administered alone at a dose of 1-100 mg/m ² per day for 1-4 days. Afterwards a patient is reevaluated, using for example, blood work and/or biopsy to determine dopamine levels and/or MRI to evaluate treatment efficacy. An HDAC inhibitor is administered, optionally, on day four of treatment plan or subsequent to the decitabine treatment if dopamine levels remain below normal or if PD symptoms persist. The treatment plan (decitabine treatment followed by HDAC inhibitor treatment) can be repeated several times or as necessary.

### Example 4

In another example, a patient suffering from fragile X syndrome is administered decitabine by intravenous injection at a dose rate of 1-100 mg/m² per day for three days. On the fourth day of treatment the patient is administered an HDAC inhibitor such as depsipeptide or Trichostatin A (TSA), which have similar potency. The depsipeptide or TSA is administered at a dose of 5-20 mg/m², preferably in a four-hour infusion. This four-day treatment course can be repeated multiple times or until FMR-1 RNA expression is upregulated.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

1. A method for treating a neurological disorder, comprising: administering to a patient suffering from the neurological disorder a therapeutically effective amount of a DNA methylation inhibitor.

2. The method of claim 1, wherein the neurological disorder is selected from the group consisting of Aarskog syndrome, Alzheimer's disease, amyotrophic lateral sclerosis (Lou Gehrig's disease), aphasia, Bell's Palsy, Creutzfeldt-Jakob disease, cerebrovascular disease, Comelia de Lange syndrome, epilepsy and other severe seizure disorders, dentatorubral-pallidoluysian atrophy, fragile X syndrome, hypomelanosis of Ito, Joubert syndrome, Kennedy's disease, Machado-Joseph's diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere's disease, Parkinson's disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William's syndrome.

3. The method of claim 1, wherein the DNA methylation inhibitor is a cytidine analog.

4. The method of claim 3, wherein the cytidine analog is decitabine or 5-azacytidine.

5. The method of claim 1, wherein the administering of the DNA methylation inhibitor is orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

6. The method of claim 1, wherein the DNA methylation inhibitor is decitabine or 5-aza-cytidine and is administered intravenously or subcutaneously.

7. The method of claim 6, wherein decitabine or 5-aza-cytidine is administered to the patient via an intravenous infusion at a dose of 1-150 mg/m² per day.

8. The method of claim 6, wherein decitabine or 5-aza-cytidine is administered to the patient via an intravenous infusion at a dose of 1-100 mg/m² per day.

9. The method of claim 6, wherein decitabine or 5-aza-cytidine is administered to the patient via an intravenous infusion at a dose of 2-50 mg/m² per day.

10. The method of claim 6, wherein decitabine or 5-aza-cytidine is administered to the patient via an intravenous infusion at a dose of 5-20 mg/m² per day.

11. The method of claim 6, wherein decitabine or 5-aza-cytidine is administered to the patient via an intravenous infusion for at least 3 days per treatment cycle at a dose of 1-100 mg/m² per day.

12. The method of claim 6, wherein decitabine or 5-aza-cytidine is administered to the patient subcutaneously at a dose of 1-100 mg/m² per day.

13. The method of claim 6, wherein decitabine or 5-aza-cytidine is administered to the patient subcutaneously at a dose of 1-50 mg/m² per day.

14. The method of claim 1, further comprising:
administering to the patient a therapeutically effective amount of a histone deacetylase inhibitor.

15. The method of claim 14, wherein the histone deacetylase inhibitor is selected from the group consisting of hydroxamic acid, cyclic peptide, benzamide, butyrate, and depudecin.

16. The method of claim 15, wherein the hydroxamic acid is selected from the group consisting of trichostatin A, suberoylanilide hydroxamic acid, oxamflatin, suberic bishydroxamic acid, m-carboxy-cinnamic acid bishydroxamic acid, valproic acid and pyroxamide.

17. The method of claim 15, wherein the cyclic peptide is selected from the group consisting of trapoxin A, apicidin and depsipeptide.

18. The method of claim 15, wherein the benzamide is MS-27-275.

19. The method of claim 15, wherein the butyrate selected from the group consisting of butyric acid, phenylbutyrate and arginine butyrate.

20. The method of claim 15, wherein the histone deacetylase inhibitor is depsipeptide and administered intravenously.

21. The method of claim 20, wherein depsipeptide is administered to a patient by continuous intravenous infusion for at least 4 hours at a dose of 1-100 mg/m².

22. The method of claim 20, wherein depsipeptide is administered to a patient by continuous intravenous infusion for at least 4 hours at a dose of 2-50 mg/m².

23. The method of claim 20, wherein depsipeptide is administered to a patient by continuous intravenous infusion for at least 4 hours at a dose of 5-25 mg/m².

24. The method of claim 20, wherein depsipeptide is administered after the administration of a DNA methylation inhibitor.

25. The method of claim 14, wherein the histone deacetylase inhibitor is phenylbutyrate and administered intravenously.

26. The method of claim 25, wherein phenylbutyrate is administered to the patient by continuous intravenous infusion for at least 2 to 3 weeks at a dose of 100-2000 mg/m² per day.

27. The method of claim 25, wherein phenylbutyrate is administered to the patient by continuous intravenous infusion for at least 2 to 3 weeks at a dose of 250-1000 mg/m² per day.

28. The method of claim 25, wherein phenylbutyrate is administered to the patient by continuous intravenous infusion for at least 2 to 3 weeks at a dose of 500-800 mg/m² per day.

29. The method of claim 14, wherein the DNA methylation inhibitor is administered prior to the administration of the histone deacetylase inhibitor.

30. A kit for treating a neurological disorder, comprising:
a first container containing decitabine or 5-azacytidine, and a second container containing a histone deacetylase inhibitor selected from the group consisting of hydroxamic acid, cyclic peptide, benzamide, butyrate, valproic acid and depudecin.

31. The kit of claim 30, wherein the hydroxamic acid is selected from the group consisting of trichostatin A, suberoylanilide hydroxamic acid, oxamflatin, suberic bishydroxamic acid, m-carboxy-cinnamic acid bishydroxamic acid and pyroxamide.

32. The kit of claim 30, wherein the cyclic peptide is selected from the group consisting of trapoxin A, apicidin and depsipeptide.

33. The kit of claim 30, wherein the benzamide is MS-27-275.

34. The kit of claim 30, wherein the butyrate is butyric acid or phenylbutyrate.

35. The kit of claim 30, further comprising an instruction for how to administer decitabine or 5-azacytidine and the histone deacetylase inhibitor for treating the neurological disorder.

36. The kit of claim 30, wherein the neurological disorder is selected from the group consisting of Aarskog syndrome, Alzheimer's disease, amyotrophic lateral sclerosis (Lou Gehrig's disease), aphasia, Bell's Palsy, Creutzfeldt-Jakob disease, cerebrovascular disease, Comelia de Lange syndrome, epilepsy and other severe seizure disorders, dentatorubral-pallidoluysian atrophy, fragile X syndrome, hypomelanosis of Ito, Joubert syndrome, Kennedy's disease, Machado-Joseph's diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere's disease, Parkinson's disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William's syndrome.

37. The kit of claim 30, wherein the neurological disorder is Lou Gehrig's disease.

38. The kit of claim 30, wherein the neurological disorder is fragile X syndrome.

39. The kit of claim 30, wherein the neurological disorder is Parkinson's disease.

40. The kit of claim 30, wherein the neurological disorder is Alzheimer's disease.

41. A method for preventing or reducing the risk of developing a neurological disorder, comprising:
administering to an individual susceptible to the neurological disorder a therapeutically effective amount of a DNA methylation inhibitor.

42. The method of claim 41, wherein the neurological disorder is selected from the group consisting of Aarskog syndrome, Alzheimer's disease, amyotrophic lateral sclerosis (Lou Gehrig's disease), aphasia, Bell's Palsy, Creutzfeldt-Jakob disease, cerebrovascular disease, Cornelia de Lange syndrome, epilepsy and other severe seizure disorders, dentatorubral-pallidoluysian atrophy, fragile X syndrome, hypomelanosis of Ito, Joubert syndrome, Kennedy's disease, Machado-Joseph's diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere's disease, Parkinson's disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William's syndrome.

43. The method of claim 41, wherein the DNA methylation inhibitor is a cytidine analog.

44. The method of claim 43, wherein the cytidine analog is decitabine or 5-azacytidine.

45. The method of claim 41, wherein the administering of the DNA methylation inhibitor is orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

46. The method of claim 41, wherein the DNA methylation inhibitor is decitabine or 5-aza-cytidine and is administered intravenously or subcutaneously.

47. The method of claim 46, wherein decitabine or 5-aza-cytidine is administered to the patient via an intravenous infusion at a dose of 1-150 mg/m² per day.

48. The method of claim 46, wherein decitabine or 5-aza-cytidine is administered to the patient via an intravenous infusion at a dose of 1-100 mg/m² per day.

49. The method of claim 46, wherein decitabine or 5-aza-cytidine is administered to the patient via an intravenous infusion at a dose of 2-50 mg/m² per day.

50. The method of claim 46, wherein decitabine or 5-aza-cytidine is administered to the patient via an intravenous infusion at a dose of 5-20 mg/m² per day.

51. The method of claim 46, wherein decitabine or 5-aza-cytidine is administered to the patient via an intravenous infusion for at least 3 days per treatment cycle at a dose of 1-100 mg/m² per day.

52. The method of claim 46, wherein decitabine or 5-aza-cytidine is administered to the patient subcutaneously at a dose of 1-100 mg/m² per day.

53. The method of claim 46, wherein decitabine or 5-aza-cytidine is administered to the patient subcutaneously at a dose of 1-50 mg/m² per day.

54. The method of claim 41, further comprising:
administering to the patient a therapeutically effective amount of a histone deacetylase inhibitor.

55. The method of claim 54, wherein the histone deacetylase inhibitor is selected from the group consisting of hydroxamic acid, cyclic peptide, benzamide, butyrate, and depudecin.

56. The method of claim 55, wherein the hydroxamic acid is selected from the group consisting of trichostatin A, suberoylanilide hydroxamic acid, oxamflatin, suberic bishydroxamic acid, m-carboxy-cinnamic acid bishydroxamic acid, valproic acid and pyroxamide.

57. The method of claim 55, wherein the cyclic peptide is selected from the group consisting of trapoxin A, apicidin and depsipeptide.

58. The method of claim 55, wherein the benzamide is MS-27-275.

59. The method of claim 55, wherein the butyrate selected from the group consisting of butyric acid, phenylbutyrate and arginine butyrate.

60. The method of claim 55, wherein the histone deacetylase inhibitor is depsipeptide and administered intravenously.

61. The method of claim 60, wherein depsipeptide is administered to a patient by continuous intravenous infusion for at least 4 hours at a dose of 1-100 mg/m².

62. The method of claim 60, wherein depsipeptide is administered to a patient by continuous intravenous infusion for at least 4 hours at a dose of 2-50 mg/m².

63. The method of claim 60, wherein depsipeptide is administered to a patient by continuous intravenous infusion for at least 4 hours at a dose of 5-25 mg/m².

64. The method of claim 60, wherein depsipeptide is administered after the administration of a DNA methylation inhibitor.

65. The method of claim 54, wherein the histone deacetylase inhibitor is phenylbutyrate and administered intravenously.

66. The method of claim 65, wherein phenylbutyrate is administered to the patient by continuous intravenous infusion for at least 2 to 3 weeks at a dose of 100-2000 mg/m² per day.

67. The method of claim 65, wherein phenylbutyrate is administered to the patient by continuous intravenous infusion for at least 2 to 3 weeks at a dose of 250-1000 mg/m² per day.

68. The method of claim 65, wherein phenylbutyrate is administered to the patient by continuous intravenous infusion for at least 2 to 3 weeks at a dose of 500-800 mg/m² per day.

69. The method of claim 54, wherein the DNA methylation inhibitor is administered prior to the administration of the histone deacetylase inhibitor.
